(19) United States
(12) Patent Application Publication
(10) Pub. No.: US 2020/0188247 A1
(43) Pub. Date: Jun. 18, 2020

(54) ANHYDROUS DEODORANT AEROSOL CONTAINING SODIUM HYDROGEN CARBONATE

(71) Applicant: Henkel AG & Co. KGaA, Duesseldorf (DE)

(72) Inventors: BERNHARD BANOWSKI, Duesseldorf (DE); IMME BREUER, Duesseldorf (DE)

(73) Assignee: Henkel AG & Co. KGaA, Duesseldorf (DE)

(21) Appl. No.: 16/695,929

(22) Filed: Nov. 26, 2019

(30) Foreign Application Priority Data
Dec. 18, 2018 (DE) 10 2018 222 056.5

(51) Int. Cl. A61K 8/25; A61Q 15/00; A61K 8/19

(52) U.S. Cl. CPC A61K 8/25; A61K 8/19; A61Q 15/00

(57) ABSTRACT

The present disclosure relates to an anhydrous cosmetic composition for controlling body odor or underarm wetness, the composition comprising an odor-absorbing substance and a thickener, the odor-absorbing substance being a water-soluble inorganic carbonate and the thickener being an amorphous silicon dioxide. Furthermore, the present disclosure relates to a cosmetic product comprising the anhydrous cosmetic composition, as well as its use.

### CROSS-REFERENCE TO RELATED APPLICATION

This application claims priority to German Patent Application No. 10 2018 222 056.5, filed Dec. 18, 2018, which is incorporated herein by reference in its entirety.

### TECHNICAL FIELD

The present disclosure relates to an anhydrous cosmetic composition for controlling body odor or underarm wetness, the composition comprising an odor-absorbing substance and a thickener, the odor-absorbing substance being a water-soluble inorganic carbonate and the thickener being an amorphous silicon dioxide.

### BACKGROUND

The washing, cleaning and care of one's own body represents a basic human need. The manufacturers of personal hygiene products are constantly trying to meet these needs of people in many ways. Especially important for daily hygiene is the prolonged elimination of body odor. Numerous specific deodorant personal care products are known in the art which have been developed for use in body regions having a high density of sweat glands, in particular in the axillary region and on the feet.

Body odor arises to a large extent from the bacterial decomposition of individual components of sweat on the skin. In body deodorization, one can roughly differentiate between active substances which absorb or cover up (fragrances, perfumes) unpleasant-smelling substances already formed, and active substances which prevent, or at least slow down the decomposition of sweat and the formation of unpleasant-smelling substances (germ-inhibiting active substances, prebiotic components and enzyme inhibitors). Depending on the chosen mechanism by which body odor is to be prevented, odor absorbers, fragrances, deodorizing ion exchangers, germ-inhibiting agents, prebiotic active components, enzyme inhibitors and other active substances can be used as cosmetic deodorizing active substances. The active substances must be included in the compositions, ensuring that the compositions can be conveniently provided in a desired dosage form and that the compositions do not cause an unpleasant sensation on the skin.

The cosmetic antiperspirants used for sweat inhibition described in the prior art contain aluminum and/or zirconium compounds as antiperspirant active substances. These antiperspirant compounds reduce sweat secretion of the body by temporarily narrowing and/or obstructing the excretory ducts of the sweat glands, so that the amount of sweat can be reduced by about 20 to about 60 percent. On the other hand, they have an additional deodorizing effect due to their antimicrobial effect. Furthermore, substances are described in the prior art that absorb already formed unpleasant-smelling substances. These comprise solids such as activated carbon, zinc ricinoleate, cyclodextrins or ion exchangers of all kinds.

Aluminum compounds, however, are especially considered critical by consumers. The desire for deodorants which are free of aluminum compounds, but very powerfully prevent the formation of odors and/or moisture, is very large. In addition, the use of the aforementioned antiperspirant compounds can be disadvantageous in that they lead to staining on clothing.

In addition to the mechanism of action, the development of deodorants and antiperspirants is directed to the dosage form. The cosmetic agents are packaged in different dosage forms, for example, as a powder, in stick form, as an aerosol spray, pump spray, liquid and gel roll-on application, cream, gel and as a soaked flexible substrate (deodorant wipes). Particularly popular with consumers are sprays containing solid particles that are present suspended in a liquid matrix. The matrix often constitutes an oil phase, often including ethers, triglycerides, ester oils or paraffinic substances.

The ingredients of sprays regularly comprise thickeners, by which the cosmetic composition is easier to use. The use of thickeners is further technically necessary since the aerosol compositions constitute suspensions that must have a certain viscosity so that the particulate active ingredients do not settle when filling and batches having different amounts of solids are obtained. A preparation of aerosol compositions, for example, with sodium hydrogen carbonate as an odor-absorbing substance in an organic solvent with a thickener, represents a challenge. By default, hydrophobically modified clay minerals are used as thickeners in such aerosol compositions. The hydrophobization is accomplished by ion exchange with quaternized organic compounds. Quaternized bentonites or hectorites modified with organic compounds develop when used with basic substances and amines (possibly at elevated temperature) that have an unpleasant odor.

### BRIEF SUMMARY

Cosmetic compositions, cosmetic products, and methods of using the same for controlling body odor and/or underarm wetness are provided. In an exemplary embodiment, a cosmetic composition is anhydrous, and includes an odor-absorbing substance and a thickener. The odor absorbing substance is a water-soluble inorganic carbonate, and the thickener is an amorphous silicon dioxide.

A cosmetic product is provided in another embodiment. The cosmetic product includes a propellant and a cosmetic composition. The propellant is a hydrocarbon, and the cosmetic composition is anhydrous and includes an odor absorbing substance and a thickener. The odor absorbing substance is a water soluble inorganic carbonate, and the thickener is an amorphous silicon dioxide. The cosmetic product includes the cosmetic composition at from about 5 to about 70 weight percent, and the propellant at from about 30 to about 95 weight percent, based on a total weight of the cosmetic product.

A method of controlling body odor or underarm wetness is provided in another embodiment. The method includes applying a cosmetic composition to the underarm, where the cosmetic composition includes an odor-absorbing substance and a thickener. The odor absorbing substance is a water-soluble inorganic carbonate, and the thickener is an amorphous silicon dioxide. The cosmetic composition is anhydrous, such that the cosmetic composition includes water at from about 0 to about 3 weight percent, based on a total weight of the cosmetic composition.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object underlying the present disclosure is to provide an aerosol composition which does not emit an amine odor. In particular, the object underlying the present disclosure is to provide a deodorant spray, in particular an odor-absorbing aerosol which contains a particulate active ingredient against body odor and a thickener, but which does not emit unpleasant odors.

The problem is solved by the subject matter of claim 1. A first subject of the present disclosure is therefore an anhydrous cosmetic composition for controlling body odor or underarm wetness, the composition containing an odor-absorbing substance and a thickener, the odor-absorbing substance being a water-soluble inorganic carbonate and the thickener being an amorphous silicon dioxide.

It has surprisingly been found that inorganic carbonates, which are predominantly basic, can be combined with thickeners based on amorphous silicon dioxide, wherein, on the one hand, a satisfactory odor-inhibiting effect is achieved, on the other hand, a sufficient viscosity can be set, wherein no amines that smell unpleasant are released.

A thickener is understood to mean a substance that makes a fluid phase more viscous after it has been added. In the present case, amorphous silicon dioxide is used, which is also referred to as solid silica.

The compositions according to the present disclosure should be anhydrous, in particular substantially anhydrous. The person skilled in the art understands an anhydrous composition as a composition to which no water is added. Due to the fact that raw materials in general—and in view of the present disclosure, in particular the particulate substances—are not completely free of water, the composition according to the present disclosure contains water in a small amount. Furthermore, products always absorb a certain degree of moisture. A preferred composition according to the present disclosure is substantially free of water. More preferably, the cosmetic preparation comprises from 0 to about 3% by weight of water, more preferably from about 0.1 to about 2% by weight, most preferably from about 0.2 to about 1% by weight, based on the total weight of the composition.

According to a preferred embodiment of the present disclosure, the water-soluble inorganic carbonate is a carbonate or hydrogen carbonate of the alkali metals or alkaline earth metals, in particular sodium hydrogen carbonate. These particulate active ingredients have proven to be advantageous in odor absorption.

According to a preferred embodiment of the present disclosure, the amorphous silicon dioxide is a hydrophobically modified amorphous silicon dioxide or a hydrophilic silicon dioxide. In particular, the hydrophobically modified amorphous silicon dioxides are suitable as excellent thickeners in anhydrous deodorants comprising basic odor-absorbing substances.

More preferably, the hydrophobically modified amorphous silicon dioxide is a silylation product of a reaction of a precipitated silica and/or a fumed silica with an organosilicon compound, the organosilicon compound preferably being an alkylsiloxane, a silane, preferably trimethylsilane, or a silazane, in particular hexamethyldisilazane. More preferably, the organosilicon compound is a compound comprising one or more alkyl groups having 1 to about 18, preferably 2 to about 14, more preferably 3 to about 12 carbon atoms. More preferably, the hydrophobically modified amorphous silicon dioxide constitutes Silica Silylate (INCI), Silica Cetyl Silylate (INCI) or Silica Dimethyl Silylate (INCI). The hydrophobically modified amorphous silicon dioxide becomes hydrophobic through the alkyl groups of the organosilicon compound. The alkyl groups are located on the surface of the silicon dioxide particles after hydrophobization. The number of carbon atoms of the alkyl group has an effect on the degree of hydrophobization. Silica Silylate (INCI), Silica Cetyl Silylate (INCI) and Silica Dimethyl Silylate (INCI) are commercially available products. A preferred example is Aerosil R 812 S.

According to a preferred embodiment of the present disclosure, the composition contains substantially no modified clay minerals, in particular no bentonites or hectorites. Modified clay minerals are hydrophobized with quaternary compounds in order to be able to be used in cosmetic deodorants. Forgoing quaternized clay minerals avoids the formation of amines.

According to a further preferred embodiment, the composition according to the present disclosure substantially contains no silicone-based emollients. Silicon-based emollients or solvents are being discussed. A forgoing of silicones is associated with the advantage of a reduced input of potentially environmentally hazardous substances.

Furthermore, according to a further preferred embodiment, the composition according to the present disclosure substantially contains no petrochemically produced emollients. The composition of the present disclosure is to substantially have no solvents or emollients conventionally produced from petrochemical sources. In the context of the present disclosure, an emollient or solvent is said to be produced as a petrochemical if, for the production of the emollient or solvent, a component derived from a petrochemical source is used in one stage. In the context of a more sustainable use of raw materials and energy, it remains desirable to use only such raw materials for cosmetic products that are accessible with the least possible use of energy from renewable raw materials.

An important aspect of the present disclosure relates to the provision of a cosmetic composition for the prevention of body odors, which is free of water-soluble aluminum and/or zirconium salts. The composition comprising the inorganic carbonate, in particular sodium hydrogen carbonate, ensures a good odor-inhibiting effect, so that said salts are dispensed with.

According to a preferred embodiment of the present disclosure, the composition further contains a second odor-absorbing substance selected from the group including cyclodextrins, zeolites, mixed salts of alkaline earth metal hydroxides and alkaline earth metal carbonates, metal hydroxides, preferably magnesium hydroxide, zinc chloride hydroxide, diatoms, silicates, preferably aluminum silicates, calcium silicate, magnesium-aluminum metasilicate, magnesium-aluminum silicate, sodium-aluminum silicate, magnesium-calcium silicate, magnesium silicate, silicon dioxides, amylodextrin, maltodextrin, bentonites, silicon dioxide, hectorite, kaolin, montmorillonite, perlite, talc, a homoglycan, preferably starch or its derivatives, aluminum or sodium salts of octenyl succinic anhydride esterified starch, modified corn starch, glyceryl starch, and mixtures of any of the foregoing. These odor-absorbing substances can form a second active substance, which is used in addition to the inorganic carbonate, in particular the sodium hydrogen carbonate.

According to a further preferred embodiment of the present disclosure, the composition is based on an organic carrier which is a cosmetic oil comprising liquid paraffin oil or isoparaffin oil, preferably isodecane, isoundecane, isododecane, isotridecane, isotetradecane, isopentadecane, isohexadecane or isoeicosane, esters of linear or branched, saturated or unsaturated C2-30 fatty alcohols with C2-30 linear or branched saturated or unsaturated fatty acids, C8-C22 fatty alcohol esters of monovalent or polyvalent C2-C7 hydroxycarboxylic acids, triethyl citrate, branched saturated or unsaturated C6-30 fatty alcohols, mono-, di- and triglycerides of linear or branched, saturated or unsaturated, optionally hydroxylated C8-30 fatty acids, dicarboxylic acid esters of linear or branched C2-C10 alkanols, addition products of ethylene oxide and/or propylene oxide onto mono- or polyvalent C3-22 alkanols, and mixtures of one or more of said organic carriers. A combination of the particulate active substances, namely the metal carbonates, in particular of the sodium hydrogen carbonate, and the hydrophobically modified amorphous silicon dioxide, in the organic carrier comprising the above substances is particularly suitable for the solution of the object underlying the present disclosure.

According to further preferred embodiments of the present disclosure, the composition comprises from about 0.1 to about 7% by weight, preferably from about 0.5 to about 4% by weight, more preferably from about 1 to about 3% by weight, of hydrophobically modified amorphous silicon dioxide; and/or the composition comprises from about 2 to about 30% by weight, preferably from about 5 to about 25% by weight, more preferably from about 10 to about 20% by weight of sodium bicarbonate; and/or the composition comprises from 0 to about 30% by weight, preferably from about 0.2 to about 20% by weight, more preferably from about 1 to about 10% by weight of the second odor-absorbing substance; and/or the composition comprises from 0 to about 10% by weight, preferably from about 0.5 to about 5, more preferably from about 1 to about 3% by weight of perfume, or from 0 to about 5% by weight, preferably from about 0.01 to about 3, more preferably from about 0.1 to about 2% by weight of volatile oil; and/or the composition comprises from about 5 to about 85% by weight, preferably from about 20 to about 70% by weight, more preferably from about 40 to about 60% by weight of cosmetic oil, in each case based on the total weight of the composition. Further components may be included in the composition in these preferred embodiments. So when an exemplary composition contains about 10% by weight of a second odor-absorbing substance, about 2% by weight of hydrophobically modified amorphous silicon dioxide, about 60% by weight of a cosmetic oil and about 2% by weight of a perfume, other components are thus included to obtain 100% by weight composition.

Further preferred deodorant active substances are selected from so-called prebiotically active components, which according to the present disclosure are to be understood to mean those components which inhibit only or at least predominantly inhibit the odor-causing germs of the skin microflora, but not the desired ones, that is, the non-odor-forming germs that belong to healthy skin microflora. Explicit are here active substances such as conifer extracts, in particular from the group of Pinaceae, and plant extracts from the group of Sapindaceae, Araliaceae, Lamiaceae and Saxifragaceae, in particular extracts of Picea spp., Paullinia sp., Panax sp., Lamium album or Ribes nigrum and mixtures to name these substances.

Further preferred deodorant active substances are selected from the germ-inhibiting perfume oils and the Deosafe® perfume oils, which are available from the company Symrise, formerly Haarmann and Reimer.

Preferably, the antiperspirant composition comprises one or more perfumes or fragrances. Fragrances or scents, in particular the fragrance and scent compounds listed below, are by definition not to be counted among the cosmetic oils. The definition of a fragrance in the context of the present application corresponds to the definition customary in the art, as can be taken from the RÖMPP Chemie Lexikon (“Römpp Lexikon Chemie”, J. Falbe, M. Regitz (ed.), 10th edition, Thieme Verlag). In the context of the present disclosure, scents or fragrances can only be considered as those which, based on the total weight of the antiperspirant composition, are present in an amount of 10% by weight and less in the antiperspirant composition. Examples of ester-type scent and fragrance compounds are benzyl acetate, phenoxyethyl isobutyrate, p-tert-butylcyclohexyl acetate, linalyl acetate, dimethylbenzylcarbinylacetate (DMBCA), phenylethylacetate, benzylacetate, ethylmethylphenylglycinate, allylcyclohexylpropionate, styrallylpropionate, benzylsalicylate, cyclohexylsalicylate, floramate, melusate and jasmecyclate. Examples of fragrance and scent compounds of the ether type are benzyl ethyl ether and ambroxane, examples of fragrance and scent compounds of the aldehyde type are the linear alkanals having 8-18 C-atoms, citral, citronellal, citronellyloxy-acetaldehyde, cyclamen aldehyde, lilial and bourgeonal, examples of fragrance and scent compounds of the ketone type are the ionones, alpha-isomethylionone and methyl cedryl ketone, examples of fragrance and scent compounds of the alcohol type are anethole, citronellol, eugenol, geraniol, linalool, phenylethyl alcohol and terpineol, examples of fragrance and scent compounds of the terpene type are limonene and pinene. Examples of fragrance and scent compounds are pine oil, citrus oil, jasmine oil, patchouly oil, rose oil, ylang-ylang oil, muscat sage oil, chamomile oil, clove oil, mint oil, cinnamon leaf oil, lime blossom oil, juniper berry oil, vetiver oil, olibanum oil, galbanum oil, labdanum oil, orange blossom oil, neroli oil, orange peel oil and sandalwood oil, further the essential oils such as angelica root oil, aniseed oil, arnica blossom oil, basil oil, bay oil, bergamot oil, champacilla oil, silver fir oil, silver fir cone oil, elemi oil, eucalyptus oil, fennel oil, pine needle oil, geranium oil, ginger grass oil, guaiac wood oil, gurdyal balm oil, helichrysum oil, ho oil, ginger oil, iris oil, cajeput oil, calamus oil, chamomile oil, camphor oil, kanga oil, cardamom oil, cassia oil, pine needle oil, copaiba balsam oil, coriander oil, spearmint oil, caraway oil, cumin oil, lavender oil, lemon grass oil, lime oil, tangerine oil, lemon balm oil, musk kernel oil, myrrh oil, clove oil, niaouli oil, orange oil, origanum oil, palmarosa oil, patchouli oil, Peruvian balsam oil, petitgrain oil, pepper oil, peppermint oil, pimento oil, pine oil, rose oil, rosemary oil, sandalwood oil, celery oil, spik oil, star aniseed oil, turpentine oil, thuja oil, thyme oil, verbena oil, juniper berry oil, wormwood oil, wintergreen oil, hyssop oil, cinnamon oil, citronella oil, lemon oil and cypress oil. Further fragrance and scent compounds are ambrettolide, alpha-amylcinnamaldehyde, anethole, anisaldehyde, anisalcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerate, borneol, bornyl acetate, alpha-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, hepticarboxylic acid methyl ester, heptaldehyde, hydroquinone dimethyl ether, hydroxycinnamaldehyde, hydroxycinnamyl alcohol, indole, iron, isoeugenol, isoeugenol methyl ether, isosafrole, jasmon, camphor, carvacrol, carvone, p-cresol methyl ether, coumarin, p-methoxyacetophenone, methyl-n-amyl ketone, methyl anthranilic acid methyl ester, p-methylacetophenone, methylchavikole, p-methylquinoline, methyl-β-naphthyl ketone, methyl-n-nonylacetaldehyde, methyl-n-nonyl ketone, muscone, β-naphthol ethyl ether, β-naphthol methyl ether, nerol, nitrobenzene, n-nonylaldehyde, nonyl alcohol, n-octylaldehyde, p-oxy-acetophenone, pentadecanolide, β-phenylethyl alcohol, phenylacetaldehyde dimethylacetal, phenylacetic acid, pulegone, safrole, salicylic acid isoamyl ester, salicylic acid methyl ester, salicylic acid hexyl ester, salicylic acid cyclohexyl ester, santalol, skatole, terpineol, thymine, thymol, γ-undecalactone, vanillin, veratrumaldehyde, cinnamaldehyde, cinnamyl alcohol, cinnamic acid, cinnamic acid ethyl ester and cinnamic acid benzyl ester. Further (more volatile) fragrances are alkyl isothiocyanates (alkyl mustard oils), butanedione, limonene, linalool, linayl acetate and propionate, menthol, menthone, methyl-n-heptenone, phellandrene, phenylacetaldehyde, terpinyl acetate, citral and citronellal. Particularly preferred, perfumes or perfume oils are present in the antiperspirant composition in an amount from about 0.05 to about 10% by weight, more preferably from about 0.1 to about 5% by weight, most preferably from about 0.5 to about 4% by weight, based on the total weight the antiperspirant composition.

The problem underlying the present disclosure is further solved by the subject matter of claim 13. A second subject of the present disclosure is therefore a cosmetic product comprising an anhydrous cosmetic composition according to the present disclosure.

In the context of the present disclosure, a cosmetic product is to be understood to mean an application-ready cosmetic product. According to a preferred embodiment of the present disclosure, the cosmetic product is an aerosol, in particular a spray. In this preferred embodiment, the anhydrous cosmetic composition according to the present disclosure is combined with a propellant to expel the anhydrous cosmetic composition from a container.

According to a preferred embodiment of the present disclosure, the cosmetic product comprises, based on the total weight of the cosmetic product, from about 5 to about 70% by weight, preferably from about 10 to about 30% by weight of the anhydrous cosmetic composition and from about 95 to about 30% by weight, preferably from about 90 to about 70% by weight of a propellant, wherein the propellant is preferably a hydrocarbon, in particular propane, butane and/or isobutane.

Alternatively, a preferred cosmetic product is a roll-on applicator. According to a further preferred embodiment, the cosmetic product comprises the anhydrous cosmetic composition which is found in a roll-on container. The cosmetic product is therefore preferably a roll-on applicator. Furthermore, in further alternatives of preferred embodiments, the cosmetic product constitutes the anhydrous cosmetic composition on a fibrous material, in particular a textile, or the cosmetic product comprises the anhydrous cosmetic composition as a cream composition.

The problem underlying the present disclosure is further solved by the subject matter of claim 15. A third subject of the present disclosure is therefore the use of an anhydrous cosmetic composition according to the present disclosure or a cosmetic product according to the present disclosure as an aerosol, as a roll-on applicator, as a cream, as a stick or on a fiber material, in particular a textile, for controlling body odor and/or underarm wetness.

Features concerning preferred embodiments of the first subject of the present disclosure, which are described above only in this regard, of course, apply correspondingly to the second subject and the third subject as features of preferred embodiments.

Very particularly preferred compositions according to the present disclosure comprise one or more of the following embodiments A) to H):

### A)

Anhydrous cosmetic composition for controlling body odor or underarm wetness, the composition comprising an odor-absorbing substance and a thickener, the odor-absorbing substance being sodium hydrogen carbonate and the thickener being a hydrophobically modified amorphous silicon dioxide.

### B)

Anhydrous cosmetic composition for controlling body odor or underarm wetness, the composition containing an odor-absorbing substance and a thickener, the odor-absorbing substance being sodium hydrogen carbonate and the thickener being a hydrophobically modified amorphous silicon dioxide, the composition being free of water-soluble aluminum and/or zirconium salts.

### C)

Anhydrous cosmetic composition for controlling body odor or underarm wetness, the composition containing an odor-absorbing substance and a thickener, the odor-absorbing substance being sodium hydrogen carbonate and the thickener being a hydrophobically modified amorphous silicon dioxide, the composition containing substantially no modified clay minerals, in particular bentonites or hectorites, containing substantially no silicone-based emollients and/or substantially no petrochemical produced emollients.

### D)

Anhydrous cosmetic composition for controlling body odor or underarm wetness, the composition containing an odor-absorbing substance and a thickener, the odor-absorbing substance being sodium hydrogen carbonate and the thickener being a hydrophobically modified amorphous silicon dioxide, the hydrophobically modified amorphous silicon dioxide being a silylation product from a reaction of a precipitated silica and/or a fumed silica with an organosilicon compound, the organosilicon compound being preferably an alkylsiloxane, a silane, preferably trimethylsilane, or a silazane, in particular hexamethyldisilazane.

### E)

Anhydrous cosmetic composition for controlling body odor or underarm wetness, the composition containing an odor-absorbing substance and a thickener, the odor-absorbing substance being sodium hydrogen carbonate and the thickener being a hydrophobically modified amorphous silicon dioxide, the hydrophobically modified amorphous silicon dioxide constituting Silica Silylate (INCI), Silica Cetyl Silylate (INCI) or Silica Dimethyl Silylate (INCI).

### F)

Anhydrous cosmetic composition for controlling body odor or underarm wetness, the composition containing an odor-absorbing substance and a thickener, the odor-absorbing substance being sodium hydrogen carbonate and the thickener being a hydrophobically modified amorphous silicon dioxide, the composition being based on an organic carrier which is a cosmetic oil, comprising liquid paraffin oil or isoparaffin oil, preferably isodecane, isoundecane, isododecane, isotridecane, isotetradecane, isopentadecane, isohexadecane or isoeicosane, esters of linear or branched saturated or unsaturated C2-30 fatty alcohols with linear or branched saturated or unsaturated C2-30 fatty acids, C8-C22 fatty alcohol esters of monovalent or polyvalent C2-C7 hydroxycarboxylic acids, triethyl citrate, branched saturated or unsaturated C6-30 fatty alcohols, mono-, di- and triglycerides of linear or branched, saturated or unsaturated saturated, optionally hydroxylated C8-30 fatty acids, dicarboxylic acid esters of linear or branched C2-C10 alkanols, addition products of ethylene oxide and/or propylene oxide on monovalent or polyvalent C3-22 alkanols, and mixtures of one or more of those mentioned.

### G)

Anhydrous cosmetic composition for controlling body odor or underarm wetness, the composition containing an odor-absorbing substance and a thickener, the odor-absorbing substance being sodium hydrogen carbonate and the thickener being a hydrophobically modified amorphous silicon dioxide, the composition being based on an organic carrier which is a cosmetic oil, the composition comprising from about 0.1 to about 7% by weight, preferably from about 0.5 to about 4% by weight, more preferably from about 1 to about 3% by weight, of hydrophobically modified amorphous silicon dioxide; from about 2 to about 30% by weight, preferably from about 5 to about 25% by weight, more preferably from about 10 to about 20% by weight, of sodium hydrogen carbonate; and from about 5 to about 85% by weight, preferably from about 20 to about 70% by weight, more preferably from about 40 to about 60% by weight, of cosmetic oil, based in each case on the total weight of the composition.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An anhydrous cosmetic composition for controlling body odor or underarm wetness, the composition comprising an odor-absorbing substance and a thickener, wherein the odor-absorbing substance is a water-soluble inorganic carbonate and the thickener is an amorphous silicon dioxide.

2. The composition according to claim 1, wherein the water-soluble inorganic carbonate is a carbonate or hydrogen carbonate of one or more of the alkali metals and the alkaline earth metals.

3. The composition according to claim 1, wherein the amorphous silicon dioxide is a hydrophobically modified amorphous silicon dioxide or a hydrophilic silicon dioxide.

4. The composition according to claim 1, wherein the composition is free of water-soluble aluminum salts and wherein the composition is free of water soluble zirconium salts.

5. The composition according to claim 3, wherein the hydrophobically modified amorphous silicon dioxide is a silylation product of a reaction of a precipitated silica and/or a fumed silica with an organosilicon compound, wherein the organosilicon compound is selected from the group of an alkylsiloxane, a silane, a silazane, or a combination thereof.

6. The composition according to claim 5, wherein the organosilicon compound comprises one or more alkyl groups which have 1 to 18 carbon atoms.

7. The composition according to claim 3, wherein the hydrophobically modified amorphous silicon dioxide is selected from the group of Silica Silylate (INCI), Silica Cetyl Silylate (INCI), Silica Dimethyl Silylate (INCI), or combinations thereof.

8. The composition according to claim 1, wherein the composition substantially comprises no modified clay minerals, substantially no silicone-based emollients and substantially no petrochemically produced emollients.

9. The composition according to claim 1, wherein the composition further comprises a second odor-absorbing substance selected from the group of cyclodextrins, zeolites, mixed salts of alkaline earth metal hydroxides and alkaline earth metal carbonates, metal hydroxides, zinc chloride hydroxide, diatoms, silicates, calcium silicate, magnesium-aluminum metasilicate, magnesium-aluminum silicate, sodium-aluminum silicate, magnesium-calcium silicate, magnesium silicate, silicon dioxides, amylodextrin, maltodextrin, bentonites, silicon dioxide, hectorite, kaolin, montmorillonite, perlite, talc, a homoglycan, aluminum or sodium salts of octenyl succinic anhydride esterified starch, modified corn starch, glyceryl starch, and mixtures of any of the foregoing.

10. The composition according to claim 1, wherein the composition comprises an organic carrier which is a cosmetic oil, wherein the cosmetic oil is selected from the group of liquid paraffin oil or isoparaffin oil, esters of linear or branched, saturated or unsaturated C2-30 fatty alcohols with linear or branched saturated or unsaturated C2-30 fatty acids, C8-C22 fatty alcohol esters of monovalent or polyvalent C2-C7 hydroxycarboxylic acids, triethyl citrate, branched saturated or unsaturated C6-30 fatty alcohols, mono-, di- and triglycerides of linear or branched, saturated or unsaturated, optionally hydroxylated C8-30 fatty acids, dicarboxylic acid esters of linear or branched C2-C10 alkanols, addition products of ethylene oxide and/or propylene oxide onto mono- or polyvalent C3-22 alkanols, and combinations thereof.

11. The composition according to claim 1, wherein the amorphous silicon dioxide is a hydrophobically modified amorphous silicon dioxide, wherein the water soluble inorganic carbonate is sodium hydrogen carbonate, and wherein the composition comprises from about 0.1 to about 7% by weight, of the hydrophobically modified amorphous silicon dioxide; from about 2 to about 30% by weight of the sodium hydrogen carbonate; from 0 to about 30% by weight of a second odor-absorbing substance; from 0 to about 10% by weight of a perfume, from 0 to about 5% by weight of a volatile oil; and from about 5 to about 85% by weight of a cosmetic oil, based in each case on a total weight of the composition.

12. A composition according to claim 1, wherein the composition is anhydrous such that the composition comprises water at from 0 to about 3 weight percent, based on a total weight of the composition.

13. The cosmetic product comprising:
a propellant, wherein the propellant is a hydrocarbon; and
a cosmetic composition, wherein the cosmetic composition comprises an odor-absorbing substance and a thickener, wherein the odor-absorbing substance is a water-soluble inorganic carbonate and the thickener is an amorphous silicon dioxide, and wherein the cosmetic composition is anhydrous; and
wherein the cosmetic product comprises the cosmetic composition at from about 5 to about 70 weight percent, and wherein the cosmetic product comprises the propellant at from about 30 to about 95 weight percent, based on a total weight of the cosmetic product.

14. The composition of claim 11 wherein:
the composition comprises the hydrophobically modified amorphous silicon dioxide at from about 1 to about 3 percent by weight, the sodium hydrogen carbonate at from about 1 to about 10 percent by weight, the second odor-absorbing substance at from about 1 to about 3 percent by weight; the perfume at from about 0.1 to about 2 percent by weight, the volatile oil at from about 0.1 to about 2 percent by weight, and the cosmetic oil at from about 40 to about 60 percent by weight, all based on the total weight of the composition.

15. A method of controlling body odor or underarm wetness, the method comprising the steps of:
applying a cosmetic composition to the underarm, wherein the cosmetic composition comprises an odor-absorbing substance and a thickener, wherein the odor-absorbing substance is a water-soluble inorganic carbonate and the thickener is an amorphous silicon dioxide, and wherein the cosmetic composition is anhydrous such that the cosmetic composition comprises water at from about 0 to about 3 weight percent, based on a total weight of the cosmetic composition.

16. The composition according to claim 1, wherein the amorphous silicon dioxide is trimethylsilane.

17. The composition according to claim 1, wherein the amorphous silicon dioxide is hexamethyldisilazane.

18. The composition according to claim 6, wherein the organosilicon compound comprises the one or more alkyl groups which have 3 to about 12 carbon atoms.

19. The composition according to claim 1, wherein the composition comprises no bentonites and no hectorites.

20. The composition of claim 11, wherein the composition is anhydrous, such that the composition comprises water at from about 0 to about 3 weight percent, based on the total weight of the composition.
